(19) United States
(12) Plant Patent Application Publication
(10) Pub. No.: US 2010/0122388 P1
(43) Pub. Date: May 13, 2010

(54) Sweetpotato CX-1 a high dry matter processing variety

(50) ;

(75) Inventor: Janice Ryan-Bohac, North Charleston, SC (US)

Correspondence Address: BARNES & THORNBURG LLP, P.O. BOX 2786, CHICAGO, IL, 60690-2786, US

(73) Assignee: CAROLINA ADVANCED RENEWABLE ENERGY, LLC, North Charleston, SC (US)

(21) Appl. No.: 12/590,776

(22) Filed: Nov. 13, 2009

Related U.S. Application Data
(60) Provisional application No. 61/114,358, filed on Nov. 13, 2008.

(51) Int. Cl. A01H 5/00

(52) U.S. Cl. PLT/258

(57) ABSTRACT

This disclosure relates to CX-1, a new and distinct variety of sweetpotato, Ipomoea batatas, (L.) Lam. It is a distinct mutant from the sweetpotato variety, Xushu 18 from China, where other superior mutant varietys of Xushu 18 have been registered. CX-1 is a distinct variety that has superior yield and higher dry matter than Xushu 18. By combining high levels of carbohydrate (mostly starch and some sugars) with high tonnage of roots per acre, CX-1 will produce some of the highest levels of ethanol per acre found in any temperate crop. This type of sweetpotato with its uniquely high dry matter, high starch content, and mealy texture can also be used to produce sweetpotato chips and fries that have significantly less oil than commercially available low dry matter sweetpotato varieties like Beauregard and have an excellent, distinctive, texture and flavor.

This application claims priority to corresponding U.S. Ser. No. 61/114,358 file Nov. 13, 2008

### LATIN NAME OF THE GENUS AND SPECIES OF THE PLANT CLAIMED

Ipomoea batatas (L.) Lam.

### VARIETY DENOMINATION

‘CX-1’

### BACKGROUND

This disclosure relates to a new and distinct variety (cultivar) of sweetpotato named CX-1.

This new variety of sweetpotato (Ipomoea batatas (L.) Lam.) was developed by selection of Xushu 18, and designated ‘CX-1.’ It was selected for large roots, very high dry matter content and higher yield. Field tests were in North Florida and South Carolina. Although there a large number of sweetpotato varieties, there is a wide range of appearance, growth parameters, and uses, not a lot is known about the genetics of this crop species. Sweetpotato is the 7^{th} largest food crop in the world and is a major food staple in many countries, mostly in the tropics.

Sweetpotatoes are underground storage roots, not tubers. The botanical description of a tuber is a short, thickened portion of an underground stem. The tuber has eyes composed of a ridge bearing a ‘scale-like’ leaf with tiny meristematic buds in the axial of this scale-like leaf. The sweetpotato is a true root, not an underground tuber that has buds. The anatomy of a storage root is the same as any root, with an additional ability to expand radially to store additional starch and nutrients. The sweetpotato crop is asexually propagated crop from ‘seed’ roots and plant cuttings.

Currently grown U.S. sweetpotato varieties were developed to be eaten as a highly nutritious and delectable vegetable. This variety type has been bred for specific traits for that use. They produce relatively uniform, attractively shaped storage roots, with dark orange flesh, a sweet, delicious flavor, and a moist texture (from 77% to 81% moisture). The typical size of these roots is from 6 to 16 ounces. The typical fresh weight yields of these varieties will range from 12 to 15 tons of marketable roots per acre.

The sweetpotato variety Xushu 18 is a variety that was publicly released in 1972 from China. It was bred by researchers from the Jiangsu Academy of Sciences in Jiangsu Province, China. Xushu 18 was a seedling from a cross from the variety Xindazi and breeding line 52-45. (Gitoner, C. 1996. Potato and Sweetpotato in China: systems, constraints, and potential. International Potato Center. Lima, Peru).

Xushu 18 has been a popular variety in China, a country that is a major producer of sweetpotatoes. Researchers in China report that during several decades, dozens of new varieties derived by mutation from named varieties have been registered. The Xushu sweetpotato Research Center in China reports that a single plant resulting from a selection of Xushu 18 with superior characteristics was named Xu 77-6 and was selected to replace normal Xushu 18 (Daifu, M., Hongmin, L., DaPeng, L., and Yi, W. 2000. Sweetpotato varieties decline in China and the present practices. International Workshop on Sweetpotato Decline Study Sep. 8-9 2000. Kyushu National Agricultural Experiment Station. Miyakonojo, Japan.)

Xushu 18 was not developed as a table vegetable. It was developed for its production of starch and for animal feed. It is not sweet or delicious, but is white fleshed, very bland and very dry,—about 28% dry matter. Commercial yields in China have been reported from 6.88 ton up to 18.5 tons of fresh root weight per acre (Gitoner, 1996). Currently there are no sweetpotato varietys, with high dry matter content, commercially grown in the U.S. for industrial uses such as starch and ethanol.

### SUMMARY

Starting with the Xushu 18, a distinctive, new variety was selected and developed for large roots, very high dry matter and higher total yield. The original Xushu 18 variety was obtained from the Plant Genetic Resourses Unit, USDA, ARS, Griffin, Ga. After years of field trials, it was determined that a selected high-yielding single plant of Xushu 18, named CX-1, produced superior yield and higher dry matter content compared to published data on Xushu 18 (Table 1).

TABLE 1

| | | | Fresh Weight in | Fresh Weight in | Root Dry |
|---|---|---|---|---|---|
| Researcher | Location | Status | Tons/ha | Tons/A | Matter % |
| Ma Daifu, CIP | China (mean | Virus Free | 40 | 17.8 | 29 |
| | 19 locations)^{u} | Virus Check | 34 | 15.2 | 28 |
| Gruneberg, Crop Sci | Peru | Mean | 30 | 13.6 | 25 |
| | Tingo Maria | Highest | 70 | 31.5 | NR^{a} |
| | La Molina | Lowest | 5 | 2.2 | NR |
| Xie Yi Zhi, ARC | Thailand | Highest | 27 | 12.0 | 26 |
| CX-1 | USA | FL | | 49.0 | 32 |
| | USA | SC | | 37.5 | 32 |

^{a}NR is Not Reported;
ha = hectors;
A = acres
Wolfgang, G., Marique, K., Zhang, D, and Hermann, M. 2005. Genptype X Environment interactions for a diverase ser of sweetpotato clones evaluated across varying ecogeographic conditions in Peru. Crop Science. 45:2160-2171.
Zhi, X. Y. Effect of Potassium of the yield of three sweet potato varieties. 1991. Asian Regional Vegetable Research Center. Proceedings of Training Workshop.
Daifu, M., Hongmin, L., DaPeng, L., and Yi, W. 2000. Sweetpotato varieties decline in China and the prevent practices. International Workshop on Sweetpotato Decline Study Sept. 8-9 2000. Kyushu National Agricultural Experiment Station. Miyakonojo, Japan.

Sweetpotato roots of CX-1 can be used to produce both starch and fuel ethanol. Estimates of at least 1500 gallons per acre of sweetpotatoes were based on field tonnage per acre, and on laboratory tests of the amount of ethanol produced per ton of dry matter from storage roots. Sweetpotato roots of CX-1 were also used to make chips and fries. CX-1 roots make excellent chips and fries compared to what can be made from the very sweet, watery texture found in vegetable types of sweetpotato like the leading U.S. variety, Beauregard, which is 19-20% dry matter. The dry matter content of the CX-1 variety is at least 32%. In contrast commercial vegetable type sweetpotatoes varietys have a dry matter content that ranges from 19 to 23%. The texture of the CX-1 was dry and mealy in contrast to vegetable types that are moist and sweet with visible syrup in the flesh. The chips made from CX-1 were crisp, delicious, and light in color, similar to a commercial chip made from the white potato. (FIG. 5). Both the CX-1 chips and fries with high dry matter take up significantly less oil and were less bitter than these products made from the white potato.

When properly cured and stored at 50° to 60° F., the CX-1 variety can store up to one year. Long-term storage is a genetically controlled trait and a requirement for any successful variety in the temperate zones of agricultural production like the U.S. This is in contrast to typical tropical dry fleshed varietys that will deteriorate very quickly in storage. The asexual reproductions run true to the original sweetpotato and to each other in all respects.

Samples of the CX-1 variety will be deposited and maintained by the Foundation Plant Services, University of California, Davis, Calif.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a photograph of a flat of rose-skinned, large roots from CX-1 sweetpotatoes.

FIG. 2 is a photograph of a very large (17 lbs.) “turnip” shaped CX-1 sweetpotato and an 8 ounce copper-skinned “Beauregard” sweetpotato (accounts for 90% of U.S. commercial market).

FIG. 3 are photographs of (A) the upper side of a CX-1 leaf; and (B) the under side of a CX-1 leaf.

FIG. 4 is a photograph looking down on a planting of CX-1 sweetpotatoes.

FIG. 5 is a photograph of chips made from CX-1 sweetpotatoes.

### DETAILED BOTANICAL DESCRIPTION

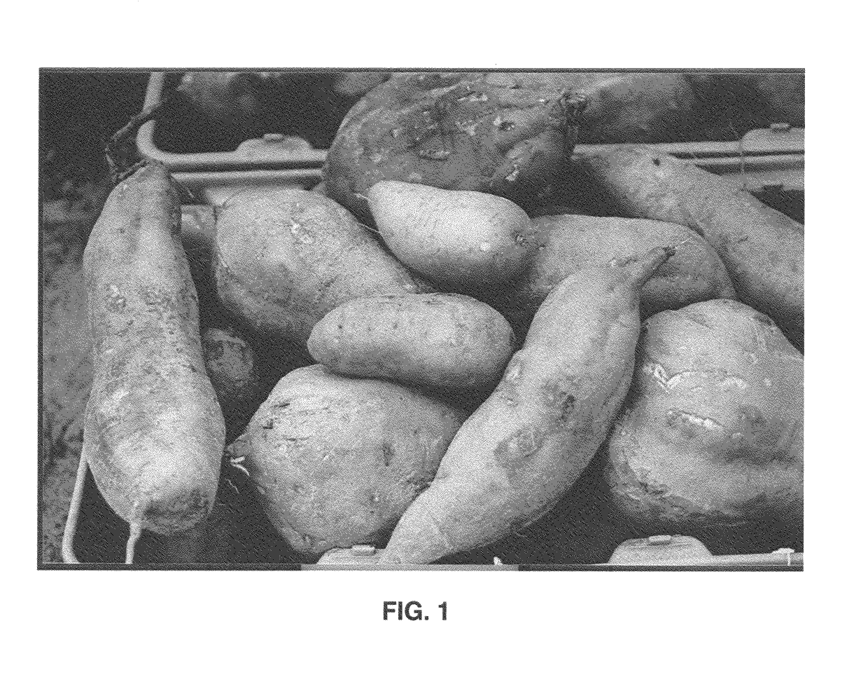

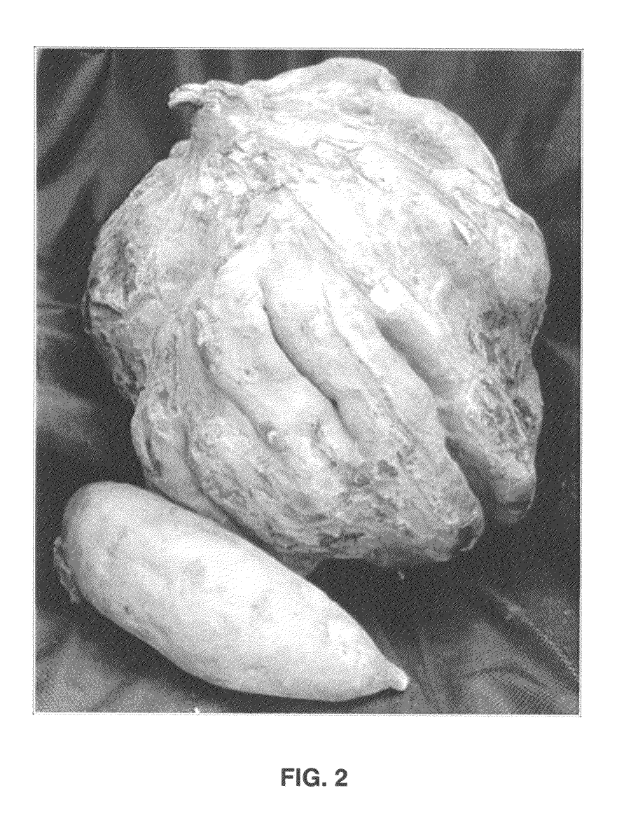

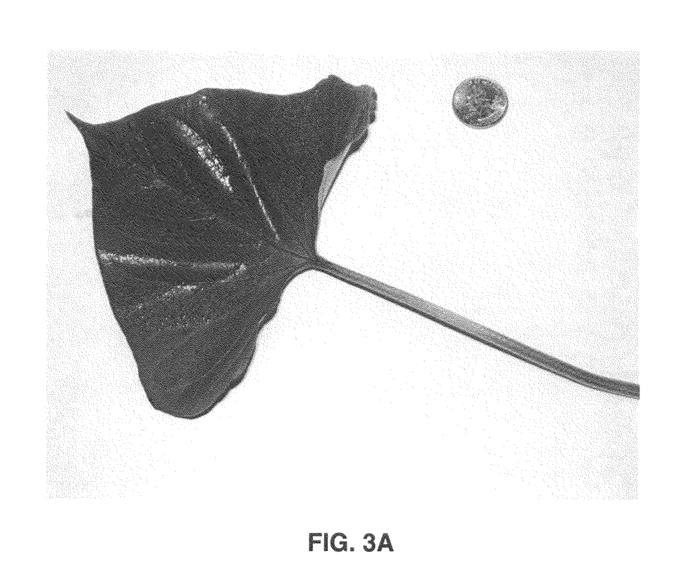

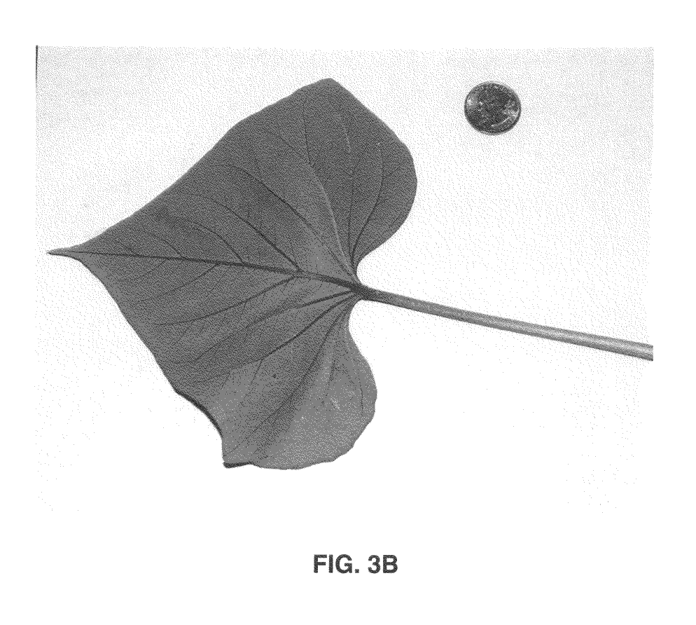

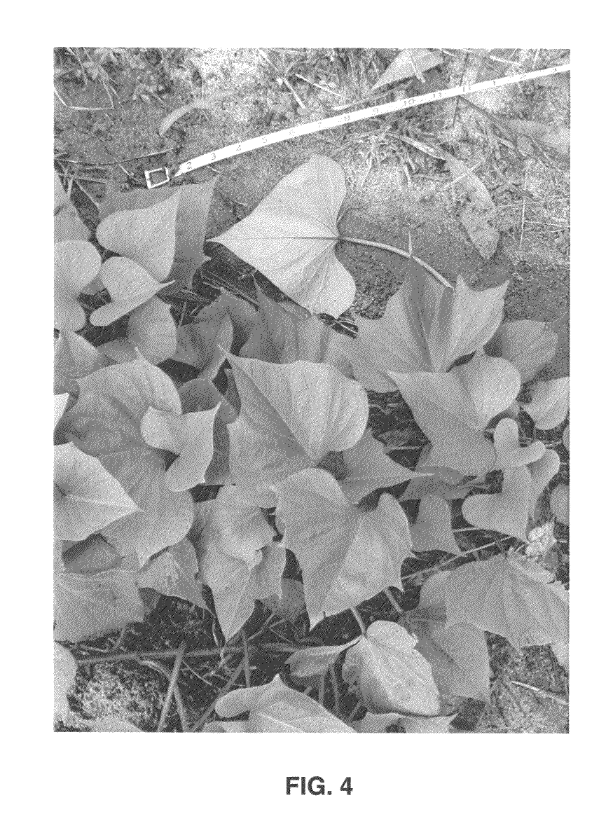

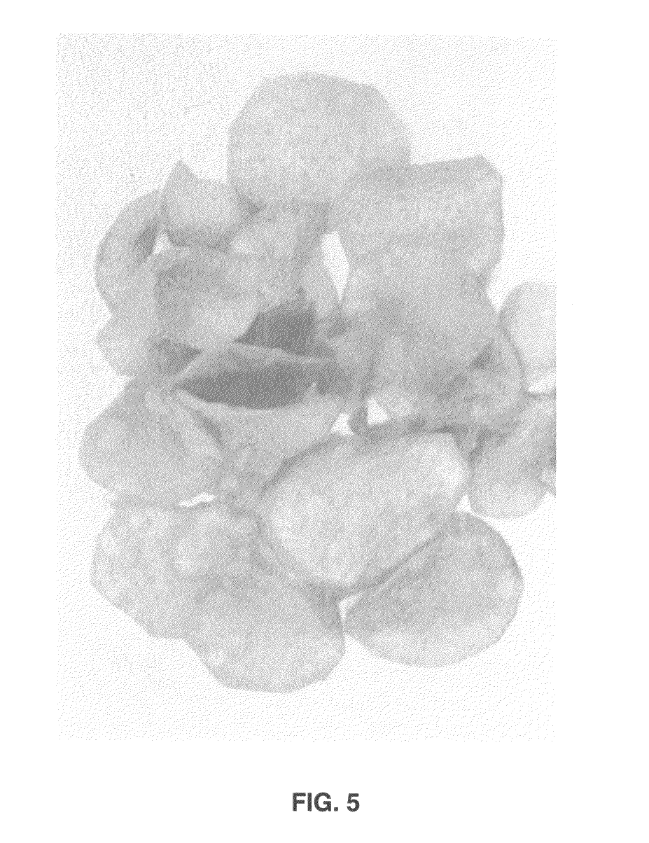

Sizes: CX-1 produces large to very large storage roots from 1 pound up to 17 lbs (FIG. 1).
Growth: The storage roots initiate at about 45 days after planting. The large roots have a smooth, oblong, blocky shape. The very large roots are deeply grooved with a ‘turnip’ type shape (FIG. 2).
Root color and texture: The storage roots have a medium rose-hued skin that is smooth and attractive on the smaller roots and is rougher and non-uniform on the very large roots. The internal flesh of the storage roots is light yellow with a uniform, starchy appearance. The color analysis of CX-1 skin and flesh was performed using the charts from Netscape entitled Netscape colors in Hue Order found in the reference (www.efg2.com/Lab/Graphics/Colors/ColorCharts.htm). The skin color is Pale Violet Red (RGB: 219, 112, 147; HSV: 340.4, 0.489, 0.859). The flesh color is Lemon Chiffon (RGB: 255, 250, 205; HSV: 54.0,0.196, 1.000).
Leaves: Leaves of the CX-1 are a medium green, cordate-shaped leaf with a purple juncture where the base of the leaf blade meets the petiole and the veins are often purple from the juncture grading to green at the leaf outer edges (FIGS. 3A and 3B).
Vines: The variety makes a vigorous vine longer and more robust than Beauregard, the leading US sweetpotato variety (FIG. 4). It is an excellent sprout producer, similar to the heirloom variety, Porto Rico.

### PRODUCTION OF THE CX-1 VARIETY

Horticultural methods were developed that differed from standard sweetpotato methods, in order to maximize the yield of CX-1. The standard spacing for vegetable sweetpotato plants is 9 inches apart in the beds, the spacing developed for CX-1 was 12 inches apart, which allowed the roots to grow larger. A Holland-type pocket transplanter was used to plant cuttings approximately 10 inches in length. All plots were watered through the transplanter, and occasionally supplemented with irrigation the first 30 days. The remainder of the season, the crop received only normal rain. In order to maximize yield, the CX-1 plants were transplanted in late April or May and allowed to grow until the vines were killed by frost. The vegetable sweetpotatoes are harvested at 90 to 100 days after planting, the CX-1 are harvested 150 to 160 days after planting (or until the first major frost).

Soil samples were submitted for soil testing. In all trials run in South Carolina and Florida, it was found that the CX-1 performed well on sandy loam soils that were low in organic matter. It is expected that the variety would perform well throughout the Coastal Plain soils that are found from southern New Jersey to Florida, and the southern counties in Alabama, Mississippi, Louisiana and East Texas. Soil tests for sweetpotato production in this type of soil recommend 90 lbs of Nitrogen, 110 lbs of Phosphorus and 110 lbs of Potash per acre. It was discovered that the highest yield of CX-1 used half of the recommended Nitrogen—45 lbs of Nitrogen per acre. Also added was recommended rate of 110 lbs per acre of Phosphorus and Potash combined with micronutrients—5 lbs of sulfur, 0.38 lbs of Boron, 10 lbs of Manganese and 0.13 lbs of Copper per acre. Also used was 1.5 pints per acre of the herbicide Dual and the grass herbicide Poast at 1 pint per acre. The insecticides sprayed were Malathion and Thionex as needed. No fungicides or nematacides were required.

Field trials were run in North Florida and in South Carolina. Storage roots of CX-1 contained 32% dry matter. The yield at 120 and 150 days after planting was found to be 12 tons to 16 tons of dry matter per acre, respectively. Samples of the sweetpotatoes were sent to the TVA laboratory in Muscle Shoals, Ala. for a chemical analysis. It was found that the CX-1 roots were approximately 32% dry matter. This dry matter was found to be 74% starch, which is readily converted to fuel ethanol in the process. Two commercially important traits make CX-1 superior to US varieties for fuel ethanol and clearly differentiate CX-1 as a new variety derived from Xushu 18. One key fact that indicates CX-1 is a stable mutant of Xushu 18, is that CX-1 roots have dramatically higher dry matter 32%. In contrast the parent clone, Xushu 18, has 27-29% dry matter. Also yield is higher in CX-1—fresh weight ranges from 37.5 to 49 tons per acre and (dry weight from 12 to 16 tons per acre). Published fresh weight data for Xushu 18 from China, Peru and Thailand have average values of 17.8, 13.6, and 12.0 tons/acre, respectively. (Daifu, M., Hongmin, L., DaPeng, L., and Yi, W. 2000. Sweetpotato varieties decline in China and the prevent practices. International Workshop on Sweetpotato Decline Study Sep. 8-9 2000. Kyushu National Agricultural Experiment Station. Miyakonojo, Japan.; Wolfgang, G., Manrique, K., Zhang, D, and Hermann, M. 2005. Genotype X Environment interactions for a diverse set of sweetpotato clones evaluated across varying ecogeographic conditions in Peru. Crop Science. 45:2160-2171; Zhi, X. Y. Effect of Potassium on the yield of three sweet potato varieties. 1991. Asian Regional Vegetable Research Center. Proceedings of Training Workshop.)

TABLE 2
A Compositional analysis of CX-1 variety

| | Test Values |
|---|---|
| Component | % of DM |
| Total Solids | 100 |
| Starch^{a} | 73.8 |
| Glucose, Maltose, Maltotriose^{b} | 0 |
| Protein | 2.45 |
| Fat | 1.86 |
| Ash (at 550° C.) | 2.89 |
| Remainder (fiber, non-starch | 19.0 |
| polysaccharides and lignin)^{c} | |

^{a}Starch content analyzed by AOAC Enzymatic analysis method Number (AOAC 979.1/AACC76.11)
^{b}Glucose, Maltose, and Maltotiose are estimated by HPLC PRAJ Standards method using Biorad Ion Exchange Column
^{c}Fiber and other organic matter is extrapolated by the difference in weight method
Woolfe, J. 1992. Sweetpotato an untapped food resource. Cambridge University Press, Cambridge, UK.

What is claimed is:

1. A new and distinct variety of the species Ipomoea batatas (L.) Lam named CX-1 substantially as described and illustrated having very high yield and high dry matter and carbohydrate content.
